(19) United States
(12) Patent Application Publication
(10) Pub. No.: US 2022/0020473 A1
(43) Pub. Date: Jan. 20, 2022

(54) METHODS AND SYSTEMS TO SENSE AND RESPOND TO MENTAL STATES

(71) Applicant: Telememory LLC, Kansas City, KS (US)

(72) Inventors: Eliot C. Arnold, Kansas City, MO (US); Martin Bukowski, Amsterdam, MO (US)

(73) Assignee: Telememory LLC, Kansas City, KS (US)

(21) Appl. No.: 17/377,345

(22) Filed: Jul. 15, 2021

Related U.S. Application Data
(60) Provisional application No. 63/051,978, filed on Jul. 15, 2020.

(51) Int. Cl. G16H 20/70; G16H 50/20; A61B 5/16; A61B 5/00

(52) U.S. Cl. CPC G16H 20/70; A61B 5/486; A61B 5/165; G16H 50/20

(57) ABSTRACT

Mechanisms are provided for responding to events which are occurring in a scene. These mechanisms translate the form of a being into a digital format, which is in turn utilized to determine the mental state of an observed being. The mechanisms, based on the state determined, take subsequent actions based on the state determined and the being metadata. The mechanisms notify any subscribed listeners of any required updates.

### CROSS-REFERENCE TO RELATED APPLICATIONS

The present application claims the benefit of U.S. Provisional Patent Application Ser. No. 63/051,978, filed Jul. 15, 2021, entitled METHOD OF DIGITAL THERAPEUTIC INTERVENTIONS TO IMPROVE PSYCHOLOGICAL WELLBEING AND OVERALL MENTAL HEALTH, which is hereby incorporated in its entirety by reference herein.

### FIELD OF THE DISCLOSURE

The field of the disclosure relates generally to computer vision and multimodal processing of sensor inputs to produce a method to detect and respond to the presence of a being.

The present invention relates generally to cognitive rehabilitation designed to improve psychological wellbeing and overall mental health of a patient using digital therapeutic techniques and interventions.

### BACKGROUND OF THE DISCLOSURE

By 2029, 42,000,000 so called Baby Boomers—including many people with cognitive impairment disorder—will require some form of long-term care (U.S. HHS). Senior living facilities development is expected to grow 21% over the next ten years to 29,700 facilities in the U.S. (Argentum). The marketplace for technology to assist aging adults in the Longevity Economy is expected to grow to nearly $30 billion in the next few years, according to the Consumer Technology Association.

Currently, there are more than 17.7 million individuals caring for someone aged 65 and older with an impairment. (National Academy of Science, Engineering and Medicine). The demands for this care giving population is only going to increase (Milken Institute) and the costs of aging is going up. By one estimate, the value of such care in 2013 totaled $470-billion. Given that by 2035 there will be 78-million individuals older than age 65 (outnumbering those under age 18 for the first time), the number of family caregivers and the demands on this group will only increase.

Personalized technology approaches to cognitive tele-rehabilitation, digital reminiscence therapy (DRT), and digital life review therapy (DLR) are limited. There are also limited non-pharmacological means of managing behavior, mood, feelings of isolation, disassociation, confusion, anxiety, and depression. Thus, there is a need in the art for a feasible means to enable healthcare providers including psychologists, psychiatrists, social workers, occupational therapists, activities therapists, and memory care facilities to evaluate and make/provide treatment recommendations seamlessly based on real time data.

### BRIEF DESCRIPTION OF THE DISCLOSURE

One aspect of the present invention concerns a system to sense and respond to the state of a being. The system translates the form of a being, in part or in whole, at a point of time or a period of time, observed, into a form which can be compared to a form of the same type or subtype. The translated state of a being is inspected using an artificial neural network, and/or other classification methods, to determine the mood and/or emotional state of the being. This information is utilized to update the state of an information system and notify any subscribed listeners.

One aspect of the present invention concerns a mobile software application running on iOS and/or Android devices is used by family members and/or care givers to setup patient profiles, select interest categories (types of art, types of music, lifestyle interests, hobbies, etc.) for those patients, invite other family members and friends to the solution, connect remote network connected devices and sensors as part of the solution to the internet, collect media from users photo galleries and social media accounts, enrich patient profile data with purchased and publicly available information, send media collected including additional commentary, meta-data, notes both text based, audio and video based to the connected electronic hardware device that initiates a media streaming and/or narrowcasting experience for the patient. The device is used to sense motion, capture facial expressions, detect objects, listen, and process audio and video images to be used in analysis of psychophysiological responses, memory recall, cognitive acuity, as well as to provide data for a recommendation engine that predicts what media to disseminate for the patient at given times. It should be understood that the present invention is not limited to the foregoing and that alternative processes may be used without departing from the spirit of the present invention.

One aspect of the present invention concerns cognitive rehabilitation achieved by way of combination of digital reminiscence and digital life review using a process including the steps of: digital biography development; memory curation; memory streaming; biometric data collection and analysis; recommendation engine; treatment report; and digital epitaph. The invention serves to elicit and engage patients by way of media curated to represent personalized desires, preferences, life stories and life events, capture the psychophysiological responses to the stimuli by way of biometric sensors and recommend future interactions based on timing and response to the media stimuli. It should be understood that the present invention is not limited to the foregoing and that alternative processes may be used without departing from the spirit of the present invention.

One aspect of the present invention concerns a method of determining an action in response to biometric data of a subject. The method includes the steps of: introducing biometric data from one or more biometric sensors to a hybrid artificial intelligence processing pipeline, where the hybrid artificial intelligence processing pipeline has one or more processors and a first current world state. Generating a plurality of observation profiles based using a processor to produce a plurality of observation profiles.

This summary is not intended to identify essential features of the present invention and is not intended to be used to limit the scope of the claims. These and other aspects of the present invention are described below in greater detail.

### BRIEF DESCRIPTION OF THE DRAWING FIGURES

Embodiments of the invention are described in detail below with reference to the attached drawing figures, wherein:

FIG. 1 is a high-level block diagram of a one example system for collection and analysis of biometric data;

FIG. 2 is a high-level block diagram of the example system environment shown in FIG. 1 with emphasis being on the elements of the hybrid artificial intelligence processing pipeline;

FIG. 3 is a high-level block diagram of a one example system for collection and analysis of biometric data;

FIG. 4a is a flow chart illustrating a method of collection and analysis of biometric data; and

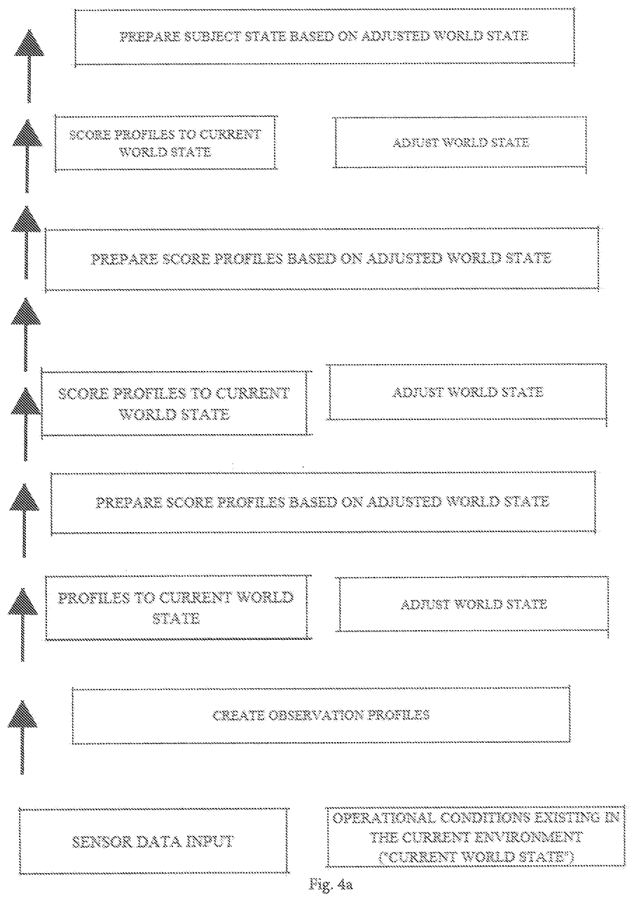

FIG. 4b is a continued part of the flow chart illustrating a method of collection and analysis of biometric data shown in FIG. 4a.

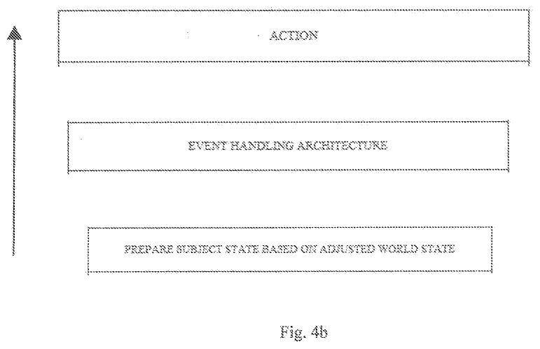

The figures are not intended to limit the present invention to the specific embodiments they depict. The drawings are not necessarily to scale. Like numbers in the Figures indicate the same or functionally similar components.

### DETAILED DESCRIPTION OF THE DISCLOSURE

The following detailed description of embodiments of the invention references the accompanying figures. The embodiments are intended to describe aspects of the invention in sufficient detail to enable those with ordinary skill in the art to practice the invention. The embodiments of the invention are illustrated by way of example and not by way of limitation. Other embodiments may be utilized, and changes may be made without departing from the scope of the claims. The following description is, therefore, not limiting. The scope of the present invention is defined only by the appended claims, along with the full scope of equivalents to which such claims are entitled.

In this description, references to “one embodiment,” “an embodiment,” or “embodiments” mean that the feature or features referred to are included in at least one embodiment of the invention. Separate references to “one embodiment,” “an embodiment,” or “embodiments” in this description do not necessarily refer to the same embodiment and are not mutually exclusive unless so stated. Specifically, a feature, component, action, step, etc. described in one embodiment may also be included in other embodiments but is not necessarily included. Thus, particular implementations of the present disclosure can include a variety of combinations and/or integrations of the embodiments described herein.

Broadly characterized, the present disclosure relates to systems and methods for collection and analysis of biometric data (i.e., a process of evaluating multimodal sensor input) relating to a subject's response to one or more stimuli during an observational period. The term “subject,” as used hereinafter, means a being, including, but not limited to a human, which is known or unknown, having an existing profile or not having an existing profile. Sensor types, from which observational data may be sourced, include, but are not limited, sensors configured to sense, capture, and/or provide visual information, infrared information, auditory information, cardiovascular information, skin conductance information, body temperature information, and neurological information. It should be understood by those having ordinary skill in the art that a wide range of sensor types may be used without departing from the spirit of the present invention. Further, a wide range of sensor configurations and arrangements are within the ambit of the present invention.

Sensor data (e.g., observation data) is introduced to a hybrid artificial intelligence processing pipeline (hereinafter referred to as “HAIPP”). As should be understood by those having ordinary skill in the art, observational data from the sensors may be introduced to the HAIPP using a wide range of means (and combinations thereof) without departing from the scope of the present invention; including, but not limited to: directly (direct connect and/or direct interconnection) ethernet; internet; IoT data protocols (e.g., hypertext transfer protocol (HTTP), hypertext transfer protocol secure (HTTPS), message queue telemetry transport (MQTT), constrained application protocol (CoAP), advanced message queuing protocol (AMQP), machine-to-machine communication protocol (M2M), extensible messaging and presence protocol (XMPP), etc.); IoT network protocols (e.g., long range wide area network (LoRaWan), bluetooth, ZigBee, etc.); peer-to-peer network (P2P); real time streaming protocol (RTSP); token ring; transmission control protocol/internet protocol (TCP/IP); user datagram protocol (UDP); wide area network (WAN); wireless application protocol (WAP); and other communications networks. In some embodiments, it may be preferrable for the observational data to be introduced to the HAIPP from the sensors directly. For instance, when the one or more sensors and the HAIPP are in close proximity to one another, as arranged components of a unitary device, a direct connection may be preferable; however, those having ordinary skill in the art will understand that the present invention is not limited to direct connection configurations for some embodiments of the present invention. In some embodiments, it may be preferrable for the observational data to be introduced to the HAIPP system from one or more sensors using TCP/IP, HTTP, HTTPS, RTSP, or combinations thereof.

In some embodiments, the HAIPP system consists of a series of processing stages, which may contain one or more processors, followed by an update function which updates the current world state. The world state is a computed value that is the system's machine knowledge of the operational conditions which exist in the environment. The computed value of the world state is, at least in part, derived from data relating to observed subjects. As will be discussed in further detail below, the value of the world state is adjusted over time as observational data is processed by the system (the “current world state”). Additionally, as will be discussed in further detail below, the value of the world state may be updated based, at least in part, on data from one or more master subject files (hereinafter referred to as “MSP”), which are periodically synchronized with a remote profile registry (hereinafter referred to as “RPR”).

An MSP may contain data such as, but not limited, to subject-specific information (e.g., age, gender, habitat, medical history, occupation, race, and other information), subject facial recognition information, subject specific models (e.g., compilations of subject-specific information and or other information concerning the subject), subject relevancy, and recent subject-related activity. When a subject is introduced to the system for the first time, a MSP is generated which produces an event to be sent to the EHA.

In some embodiments, the HAIPP system includes an initial stage. In such embodiments, the initial stage may include one or more buffers, the current world state, and one or more processors. As used herein, the term “buffer” generally refers to a connection handler, which reads frames from an open communications channel, appending the frames to a memory area where data is temporarily stored until it is no longer relevant for use by HAIPP. In one embodiment, the initial stage executes the one or more processors against inputs from the one or more buffers (observational data from each sensor being monitored) and one or more inputs from the world state to produce data which relates to the state of the environment, identification of a subject, or some other state. In one embodiment, the one or more processors of the initial stage generate one or more observation profiles based, at least in part, on the inputs from the one or more buffers and the one or more inputs from the world state (the observation profiles are hereinafter referred to as “OP”). OPs may include, but are not limited to, movement profiles, pose profiles, gaze profiles, facial embedding profiles, and eye profiles, identified sound profiles, unidentified sound profiles, and sound timber profiles. In one embodiment, initial stage processors include an update function that informs the world state of information contained in the observation data, including, but not limited to, one or more OPs.

In some embodiments, the HAIPP includes a consecutive stage. This consecutive stage includes one or more models that determine and generate one or more subject score profiles based, at least in part, on the one or more inputs from the current world state as manipulated based, at least in part, on data from the initial stage. In some embodiments, subject score profiles may include, but are not limited to, subject vocalizations, subject movement, subject attention, and/or subject affect. In some embodiments, the model of this consecutive stage is selected based, at least in part, on information concerning the current MSP from the RPR. More particularly, in such embodiments, the selection of the one or more models is, at least in part, based on subject-specific variables including, but not limited to, age, medical history, occupation, race, regional, gender and/or custom subject-specific instances. In some embodiments, models of this consecutive stage include an update function that informs the world state of information, including, but not limited to, subject score profiles, which provides updated current world state.

In some embodiments, the HAIPP system includes another consecutive stage. This consecutive stage includes one or more models that determine and generate one or more subject states based, at least in part, on the one or more inputs from the current world state as manipulated based, at least in part, on data from other stages. Subject states may include mental states and/or other states including, but not limited to, attentiveness, mood, joy, loneliness, anxiety, and depression. In some embodiments, the model of this consecutive stage is selected based, at least in part, on information concerning the current MSP from the RPR. More particularly, in such embodiments, the selection of the one or more models is, at least in part, based on subject-specific variables including, but not limited to, age, medical history, occupation, race, regional, gender and/or custom subject-specific instances. In some embodiments, models of this consecutive stage includes an update function that informs the world state of information, including, but not limited to, subject states, which provides updated current world state.

Following the execution of the HAIPP, the current world state is programmatically analyzed using techniques which may include, but are not limited to, decision trees, reinforcement learning, and error handling. The output of this programmatic analysis is zero or more events. Events are known serializable, binary, mixed, or other structures of data which can be sent and utilized by other parts of an information system. These events may be transmitted to a recommendation engine, such as an event handling architecture (hereinafter referred to as “EHA”): directly or via an intermediary system; immediately, or, if immediate transmission is not possible and/or desirable, then the event may be stored in a memory space for later transmission; and by itself or along with a batch of events. EHA is an enterprise system which takes in events and catalogues these events into various information systems, which may include, but are not limited to, time series databases, object storage, distributed databases, caches, and data warehouses. As should be understood by those having ordinary skill in the art, the events may be transmitted to the EHA using a wide range of means (and combinations thereof) without departing from the scope of the present invention; including, but not limited to: cellular network; direct connect; direct interconnection; ethernet; internet; IoT data protocols (e.g., hypertext transfer protocol (HTTP), hypertext transfer protocol secure (HTTPS), message queue telemetry transport (MQTT), constrained application protocol (CoAP), advanced message queuing protocol (AMQP), machine-to-machine communication protocol (M2M), extensible messaging and presence protocol (XMPP), etc.); IoT network protocols (e.g., long range wide area network (LoRaWan), bluetooth, ZigBee, etc.); local area network (LAN); peer-to-peer network (P2P); real time streaming protocol (RTSP); token ring; transmission control protocol/internet protocol (TCP/IP); user datagram protocol (UDP); wide area network (WAN); Wi-Fi; and/or wireless application protocol (WAP). In one embodiment, it is preferable for the transmission to be secured using standard encryption.

When an event is taken in, these events are catalogued into various information systems, which may include, but are not limited to, time series databases, object storage, distributed databases, and caches. The EHA makes programmatic determinations on what actions should be taken for an event, or a series of events, using various information processing techniques; such as, but not limited to stateful stream processing, stateless stream processing, and batch processing. The programmatic determinations of actions made by the EHA may include, but are not limited, to sending notifications to an app (e.g., mobile app, web app, etc.), compiling and transmitting a report, executing actions on a device, sending email, sending text messages, making updates to a MSP, initiation of model training sessions, invoking an internal or external application programming interface (API), or storing information into various information systems, which may include, but are not limited to, relational databases, object storage, distributed databases, caches, and data warehouses.

In some embodiments, EHA action produces a report that includes determinations concerning the subject's cognitive well-being during the most recent observation period and, if applicable, the subject's cognitive well-being based on multiple observation sessions. The EHA action may also include automated transmission of the report to any subscribed user authorized to receive such a report; such as healthcare providers, caregivers, family members, and the like. Information in the report may be used by a healthcare provider to diagnose and treat respective patients. EHA action reports may also include diagnostic determinations and/or treatment determinations to facilitate licensed healthcare providers with respect to treatment of the subject.

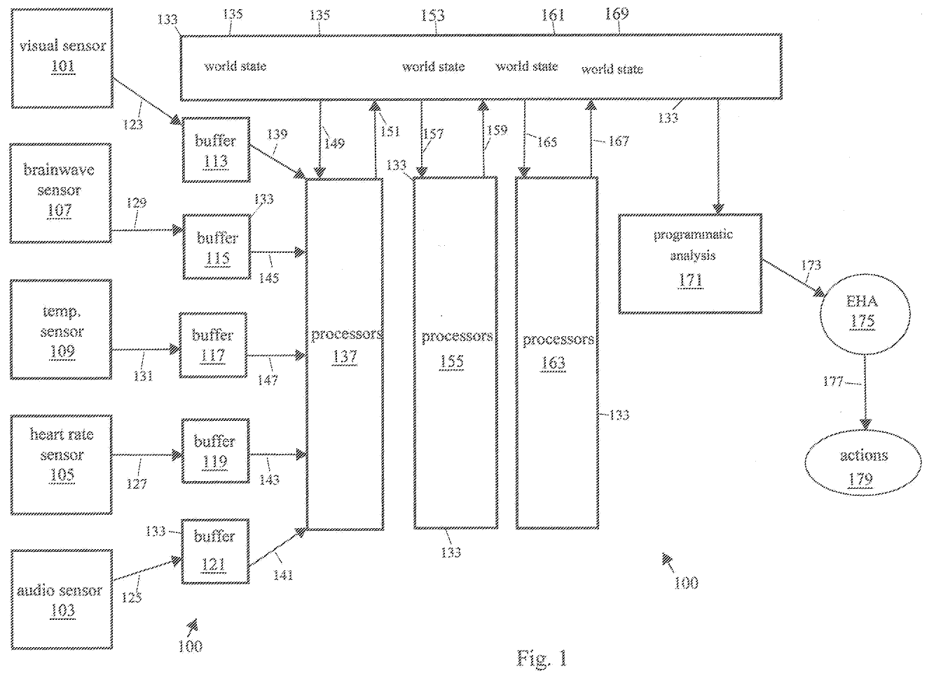

An example system environment according to certain embodiments of the present invention is shown in FIG. 1. System 100 includes a plurality of sensors arranged in proximity to the subject (not shown) to capture observational data of the subject's response to one or more stimuli during an observational period. The sensors of system 200 include a visual sensor 101, an audio sensor 103, a heartrate sensor 105, a brainwave sensor 107, and a body temperature sensor 109. As depicted in FIG. 1, each of the sensors 101-109 provide a corresponding buffer 113-121 with observational data indicated as follows: visual sensor 101 providing visual data 123; audio sensor 103 providing audio data 125; heartrate sensor 105 providing cardiovascular data 127; brainwave sensor 107 providing neurological data 129; and body temperature sensor 109 providing temperature data 131, of the subject during the observational period.

Data captured by each of the sensors, 101-109, is introduced to HAIPP 133. The initial stage of HAIPP 133 includes one or more buffers 113-121, the current world state 135, and one or more processors 137. The initial stage of HAIPP 133 executes the one or more processors 137 against inputs 139-147 from the one or more buffers 113-121 and one or more inputs 149 from the world state 135 to produce data which relates to the state of the environment, identification of a subject, or some other state. The one or more processors 137 of the initial stage generate one or more OPs, at least in part, on the inputs from the one or more buffers 139-147 and the one or more inputs 149 from the world state 135. The OPs include movement profiles, pose profiles, gaze profiles, facial embedding profiles, and eye profiles, identified sound profiles, unidentified sound profiles, and sound timber profiles, which are represent as a single OP arrow in FIG. 1 (i.e., 147). Processors 137 include an update function 153 that informs the current world state 135 of information contained in the observation data, including, but not limited to, the one or more OPs, which provides updated current world state 153.

HAIPP 133 includes a consecutive stage. This consecutive stage includes one or more processors 155 that determine and generate one or more subject score profiles based, at least in part, on the one or more inputs 157 from the current world state 153 as manipulated based, at least in part, data from the initial stage. The subject score profiles may include subject vocalizations, subject movement, subject attention, and/or subject affect. In some embodiments, the processor model of this consecutive stage is selected based, at least in part, on information concerning the current MSP from the RPR. More particularly, in such embodiments, the selection of the one or more processors is, at least in part, based on subject-specific variables including, but not limited to, subject age, medical history, occupation, race, regional, sex, and/or custom subject-specific instances. Processors 155 of this consecutive stage include an update function 159 that informs world state 153 of information, including, but not limited to, subject score profiles, which provides updated current world state 161.

HAIPP system 133 includes another consecutive stage. This consecutive stage includes one or more processors 163 that determine and generate one or more subject states based, at least in part, on the one or more inputs 165 from the current world state 161 as manipulated based, at least in part, on data from other stages. Subject states may include mental states and/or other states including, but not limited to, attentiveness, mood, joy, loneliness, anxiety, and depression. Processors 163 of this consecutive stage include an update function 167 that informs the world state of information, including, but not limited to, subject states, which provides updated current world state 169.

Following the execution of HAIPP 133, the current world state 169 is programmatic analysis 171 using techniques which may include, but are not limited to, decision trees, reinforcement learning, and error handling. The output of programmatic analysis is zero or more events 173. Events 173 are known serializable, binary, mixed, or other structures of data which can be sent and utilized by other parts of an information system. These events 173 are transmitted to EHA 175. EHA 175 is an enterprise system which takes in events and/or catalogued events from various information systems, which may include, but are not limited to, time series databases, object storage, distributed databases, caches, and data warehouses. EHA 175 makes programmatic determinations 177 on what actions 179 should be taken for an event, or a series of events, using various information processing techniques; such as, but not limited to stateful stream processing, stateless stream processing, and batch processing. Actions 179 may include, but are not limited, to sending notifications to an app (e.g., mobile app, web app, etc.), compiling and transmitting a report, executing actions on a device, sending email, sending text messages, making updates to a MSP, initiation of model training sessions, invoking an internal or external application programming interface (API), or storing information into various information systems, which may include, but are not limited to, relational databases, object storage, distributed databases, caches, and data warehouses.

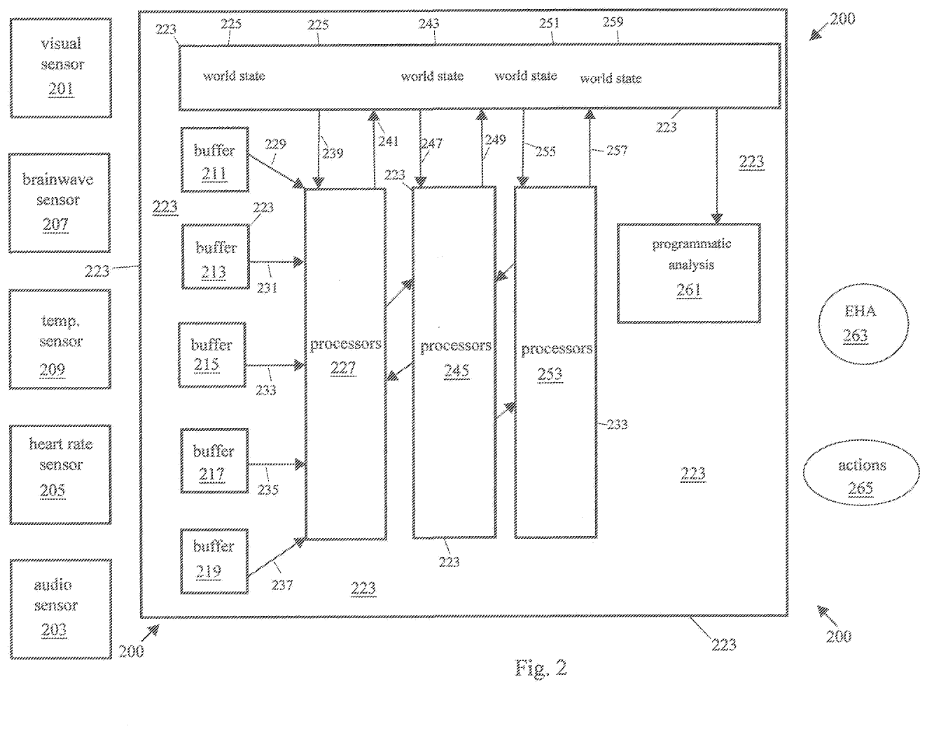

An example system environment according to certain embodiments of the present invention is shown in FIG. 2. System 200 includes a plurality of sensors arranged in proximity to the subject (not shown) to capture observational data of the subject's response to one or more stimuli during an observational period. The sensors of system 200 include a visual sensor 201, an audio sensor 203, a heartrate sensor 205, a brainwave sensor 207, and a body temperature sensor 209.

Data captured by each of the sensors, 201-209, is introduced to HAIPP 123. The initial stage of HAIPP 223 includes one or more buffers 113-121, the current world state 225, and one or more processors 227. The initial stage of HAIPP 223 executes the one or more processors 227 against inputs 229-237 from the one or more buffers 213-119 and one or more inputs 139 from the world state 225 to produce data which relates to the state of the environment, identification of a subject, or some other state. The one or more processors 227 of the initial stage generate one or more OPs, at least in part, on the inputs from the one or more buffers 229-237 and the one or more inputs 239 from the world state 225. The OPs include movement profiles, pose profiles, gaze profiles, facial embedding profiles, and eye profiles, identified sound profiles, unidentified sound profiles, and sound timber profiles, which are represent as a single OP arrow in FIG. 2 (i.e., 237). Processors 227 include an update function 243 that informs the current world state 225 of information contained in the observation data, including, but not limited to, the one or more OPs, which provides updated current world state 243.

HAIPP 223 includes a consecutive stage. This consecutive stage includes one or more processors 245 that determine and generate one or more subject score profiles based, at least in part, on the one or more inputs 247 from the current world state 243 as manipulated based, at least in part, data from the initial stage. The subject score profiles may include subject vocalizations, subject movement, subject attention, and/or subject affect. In some embodiments, the processor model of this consecutive stage is selected based, at least in part, on information concerning the current MSP from the RPR. More particularly, in such embodiments, the selection of the one or more processors is, at least in part, based on subject-specific variables including, but not limited to, subject age, medical history, occupation, race, regional, sex, and/or custom subject-specific instances. Processors 245 of this consecutive stage include an update function 249 that informs world state 243 of information, including, but not limited to, subject score profiles, which provides updated current world state 251.

HAIPP system 223 includes another consecutive stage. This consecutive stage includes one or more processors 243 that determine and generate one or more subject states based, at least in part, on the one or more inputs 255 from the current world state 251 as manipulated based, at least in part, on data from other stages. Subject states may include mental states and/or other states including, but not limited to, attentiveness, mood, joy, loneliness, anxiety, and depression. Processors 253 of this consecutive stage include an update function 257 that informs the world state of information, including, but not limited to, subject states, which provides updated current world state 259.

Following the execution of HAIPP 223, the current world state 169 is programmatic analysis 261 using techniques which may include, but are not limited to, decision trees, reinforcement learning, and error handling. The output of programmatic analysis is zero or more events (not shown). Events are known serializable, binary, mixed, or other structures of data which can be sent and utilized by other parts of an information system. These events are transmitted to EHA 263. EHA 263 is an enterprise system which takes in events and/or catalogued events from various information systems, which may include, but are not limited to, time series databases, object storage, distributed databases, caches, and data warehouses. EHA 263 makes programmatic determinations (not shown) on what actions 265 should be taken for an event, or a series of events, using various information processing techniques; such as, but not limited to stateful stream processing, stateless stream processing, and batch processing. Actions 265 may include, but are not limited, to sending notifications to an app (e.g., mobile app, web app, etc.), compiling and transmitting a report, executing actions on a device, sending email, sending text messages, making updates to a MSP, initiation of model training sessions, invoking an internal or external application programming interface (API), or storing information into various information systems, which may include, but are not limited to, relational databases, object storage, distributed databases, caches, and data warehouses.

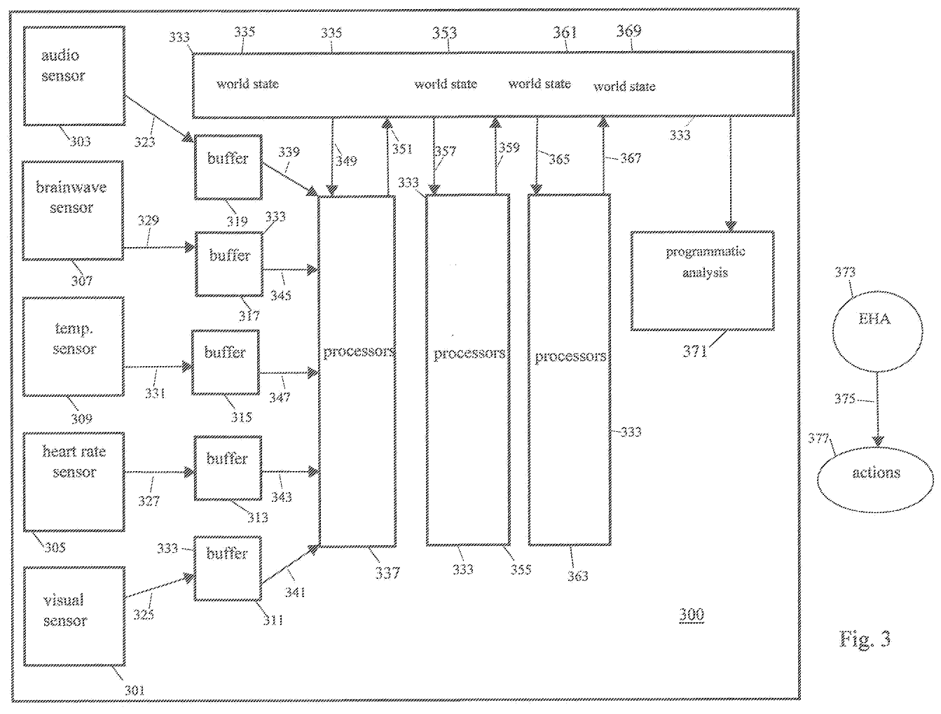

An example system environment according to certain embodiments of the present invention is shown in FIG. 3. System 300 includes a plurality of sensors arranged in proximity to the subject (not shown) to capture observational data of the subject's response to one or more stimuli during an observational period. The sensors of system 300 include a visual sensor 301, an audio sensor 303, a heartrate sensor 305, a brainwave sensor 307, and a body temperature sensor 309. As depicted in FIG. 3, each of the sensors 301-309 provide a corresponding buffer 313-319 with observational data indicated as follows: visual sensor 301 providing visual data 323; audio sensor 303 providing audio data 325; heartrate sensor 305 providing cardiovascular data 327; brainwave sensor 307 providing neurological data 329; and body temperature sensor 309 providing temperature data 331, of the subject during the observational period.

Data captured by each of the sensors, 301-309, is introduced to HAIPP 333. The initial stage of HAIPP 333 includes one or more buffers 313-319, the current world state 335, and one or more processors 337. The initial stage of HAIPP 333 executes the one or more processors 337 against inputs 339-347 from the one or more buffers 313-319 and one or more inputs 349 from the world state 335 to produce data which relates to the state of the environment, identification of a subject, or some other state. The one or more processors 337 of the initial stage generate one or more OPs, at least in part, on the inputs from the one or more buffers 339-147 and the one or more inputs 349 from the world state 335. The OPs include movement profiles, pose profiles, gaze profiles, facial embedding profiles, and eye profiles, identified sound profiles, unidentified sound profiles, and sound timber profiles, which are represent as a single OP arrow in FIG. 3 (i.e., 347). Processors 337 include an update function 353 that informs the current world state 335 of information contained in the observation data, including, but not limited to, the one or more OPs, which provides updated current world state 353.

HAIPP 333 includes a consecutive stage. This consecutive stage includes one or more processors 355 that determine and generate one or more subject score profiles based, at least in part, on the one or more inputs 357 from the current world state 353 as manipulated based, at least in part, data from the initial stage. The subject score profiles may include subject vocalizations, subject movement, subject attention, and/or subject affect. In some embodiments, the processor model of this consecutive stage is selected based, at least in part, on information concerning the current MSP from the RPR. More particularly, in such embodiments, the selection of the one or more processors is, at least in part, based on subject-specific variables including, but not limited to, subject age, medical history, occupation, race, regional, sex, and/or custom subject-specific instances. Processors 355 of this consecutive stage include an update function 359 that informs world state 353 of information, including, but not limited to, subject score profiles, which provides updated current world state 361.

HAIPP system 333 includes another consecutive stage. This consecutive stage includes one or more processors 363 that determine and generate one or more subject states based, at least in part, on the one or more inputs 365 from the current world state 361 as manipulated based, at least in part, on data from other stages. Subject states may include mental states and/or other states including, but not limited to, attentiveness, mood, joy, loneliness, anxiety, and depression. Processors 363 of this consecutive stage include an update function 367 that informs the world state of information, including, but not limited to, subject states, which provides updated current world state 369.

Following the execution of HAIPP 333, the current world state 369 is programmatic analysis 371 using techniques which may include, but are not limited to, decision trees, reinforcement learning, and error handling. The output of programmatic analysis is zero or more events (not shown). Events are known serializable, binary, mixed, or other structures of data which can be sent and utilized by other parts of an information system. These events are transmitted to EHA 373. EHA 373 is an enterprise system which takes in events and/or catalogued events from various information systems, which may include, but are not limited to, time series databases, object storage, distributed databases, caches, and data warehouses. EHA 373 makes programmatic determinations 375 on what actions 377 should be taken for an event, or a series of events, using various information processing techniques; such as, but not limited to stateful stream processing, stateless stream processing, and batch processing. Actions 377 may include, but are not limited, to sending notifications to an app (e.g., mobile app, web app, etc.), compiling and transmitting a report, executing actions on a device, sending email, sending text messages, making updates to a MSP, initiation of model training sessions, invoking an internal or external application programming interface (API), or storing information into various information systems, which may include, but are not limited to, relational databases, object storage, distributed databases, caches, and data warehouses.

Any actions, functions, operations, and the like recited herein may be performed in the order shown in the figures and/or described above or may be performed in a different order. Furthermore, some operations may be performed concurrently as opposed to sequentially. Although the methods are described above, for the purpose of illustration, as being executed by an example system and/or example physical elements, it will be understood that the performance of any one or more of such actions may be differently distributed without departing from the spirit of the present invention.

The term “processor,” and the like, as used herein, means one or more computer models which take an input of a specific structure and produce an output of a specific structure, unless expressly specified otherwise herein. All terms defined herein in the singular shall have a comparable meaning when used in the plural and vice versa.

The term “network,” “communications network,” and the like, as used herein, may, unless otherwise stated, broadly refer to substantially any suitable technology for facilitating communications (e.g., GSM, CDMA, TDMA, WCDMA, LTE, EDGE, OFDM, GPRS, EV-DO, UWB, WiFi, IEEE 802 including Ethernet, WiMAX, and/or others), including supporting various local area networks (LANs), personal area networks (PAN), or short-range communications protocols.

The term “communication component,” “communication interface,” and the like, as used herein, may, unless otherwise stated, broadly refer to substantially any suitable technology for facilitating communications, and may include one or more transceivers (e.g., WWAN, WLAN, and/or WPAN transceivers) functioning in accordance with IEEE standards, 3GPP standards, or other standards, and configured to receive and transmit signals via a communications network.

The term “memory area,” “storage device,” and the like, as used herein, may, unless otherwise stated, broadly refer to substantially any suitable technology for storing information, and may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others.

Although the invention has been described with reference to the one or more embodiments illustrated in the figures, it is understood that equivalents may be employed, and substitutions made herein without departing from the scope of the invention as recited in the claims.

What is claimed is:

1. A method of determining an action in response to biometric data of a subject, said method comprising the steps of:
(a) receiving, at a hybrid artificial intelligence processing pipeline (hereinafter referred to as “HAIPP”), biometric data from one or more biometric sensors, wherein said HAIPP comprises one or more processors and a first current world state;
(b) generating, by the one or more processors, a plurality of observation profiles based, at least in part, on the biometric data and a data input of the first world state;
(c) generating, by the one or more processors, a second current world state based, at least in part, on an input of the plurality of observation profiles to the first world state;
(d) generating, by the one or more processors, one or more subject score profiles based, at least in part, on a data input of the second current world state;
(e) generating, by the one or more processors, a third current world state based, at least in part, on an input of the one or more subject score profiles to the second current world state;
(f) generating, by the one or more processors, one or more subject states based, at least in part, on a data input of the third current world state;
(g) generating, by the one or more processors, a fourth current world state based, at least in part, on an input of the one or more subject states to the third current world state;
(h) generating, by the one or more processors, an event based, at least in part, on data of the fourth current world state; and
(i) determining, by an event handling architecture, at least one action to take in response to data of the event.
